(19) United States
(12) Patent Application Publication
(10) Pub. No.: US 2005/0281767 A1
(43) Pub. Date: Dec. 22, 2005

(54) Antiperspirant compositions comprising ozokerite

(76) Inventors: David William Walling, Cincinnati, OH (US); Phi Van Chu, Cincinnati, OH (US); Robert John Elsbrock, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/158,170

(22) Filed: Jun. 21, 2005

Related U.S. Application Data
(60) Provisional application No. 60/581,645, filed on Jun. 21, 2004.

(51) Int. Cl. A61K 7/32

(52) U.S. Cl. 424/65

(57) ABSTRACT

Anhydrous antiperspirant compositions comprising a primary structurant, such as ozokerite, a secondary structurant, and an antiperspirant active. The present invention also relates to methods of using such compositions.

### CROSS REFERENCE TO RELATED APPLICATION

This application claims the benefit of U.S. Provisional application Ser. No. 60/581,645 (Case 9677P), filed on Jun. 21, 2004.

### FIELD

The present invention relates to anhydrous antiperspirant compositions comprising a primary structurant, such as ozokerite, a secondary structurant, and an antiperspirant active. The present invention relates to anhydrous antiperspirant compositions for application to human skin, especially the axilla.

### BACKGROUND

There are many types of antiperspirant compositions that are commercially available or otherwise known in the antiperspirant art. These products typically contain an antiperspirant active, e.g. zirconium or aluminum salts or combinations thereof, solubilized or dispersed in a suitable liquid carrier, and sufficient structurants to produce the desired solid form in cases of sticks and the desired rheological character in cases of creams.

Antiperspirant compositions are designed to provide effective perspiration and odor control while also being cosmetically acceptable during and after application onto the underarm area of the skin. However, the choice of structurant and level of structurant used in the product can have negative effects on the product's antiperspirant efficacy and application aesthetics. Thus, the need still exists for structurant systems that provide improved effects on antiperspirant efficacy and application aesthetics.

### SUMMARY

The present invention relates to anhydrous antiperspirant compositions comprising a primary structurant, such as ozokerite, a secondary structurant, and an antiperspirant active. Specifically, the present invention relates to antiperspirant compositions comprising a primary structurant, such as ozokerite wax, or some other suitable petroleum wax; a secondary structurant, such as a non-petroleum wax or structurant; and an antiperspirant active. This composition has improved antiperspirant efficacy along with improved consumer application aesthetics. The present invention also relates to methods of using such compositions.

### DETAILED DESCRIPTION

While the specification concludes with the claims particularly pointing and distinctly claiming the invention, it is believed that the present invention will be better understood from the following description.

The antiperspirant compositions of the present invention utilize a dual structurant system, wherein the primary structurant is a petroleum wax, such as ozokerite wax, and the secondary structurant is chosen from a group of common non-petroleum wax based antiperspirant structurants. It has been found that use of the dual structurant system results in an antiperspirant product with improved antiperspirant efficacy, superior application aesthetics, and an improved surface appearance. Thus, compositions of the present invention are useful for improving the antiperspirant efficacy and consumer application properties of antiperspirant products. Further, the compositions of the present invention also provide improved stick appearance, such as a higher surface gloss.

The antiperspirant compositions of the present invention comprise a primary structurant, such as ozokerite and other petroleum waxes, a secondary structurant, and an antiperspirant active. The compositions may further comprise liquid carriers and additional components. Each is described in detail hereinafter.

All percentages, parts and ratios are based upon the total weight of the antiperspirant compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “antiperspirant compositions” are those compositions that are applied in a thin film to the axilla area in order to reduce or eliminate underarm perspiration. Products contemplated by the phrase “antiperspirant composition” include, but are not limited to, liquids (e.g., aerosols, pump sprays, roll-ons), solids (e.g., gel solids, invisible solids, wax solid sticks), semi-solids (e.g. creams, soft solids, lotions), and the like, provided that the selected form contains all the essential elements as defined herein.

The term “ambient conditions,” as used herein, refers to surrounding conditions under about one atmosphere of pressure, at about 50% relative humidity, and at about 25° C., unless otherwise specified.

The term “anhydrous” as used herein means that the antiperspirant stick composition of the present invention, and the essential or optional components thereof, are substantially free of added or free water. From a formulation standpoint, this means that the anhydrous antiperspirant stick compositions of the present invention contain less than about 1%, and more specifically zero percent, by weight of free or added water, other than the water of hydration typically associated with the particulate antiperspirant active prior to formulation.

The term “structurant”, as used herein, means any material known or otherwise effective in providing suspending, gelling, viscosifying, solidifying and/or thickening properties to the composition, or those materials which otherwise provide structure to the final product form. These solid structurants include one or more solid crystalline or other nonpolymeric suspending agents suitable for topical application to human skin. Suitable suspending agents are those that can form in the composition a crystalline or other matrix within which volatile solvents, non-volatile solvents, or other liquid components of the composition are contained. Such materials will typically be solids under ambient conditions and include organic solids, waxes, crystalline or other gellants, or combinations thereof. A structurant provides a uniform distribution of the particulate active throughout the product and also controls product hardness or rheology.

The term “particulate,” as used herein, refers to compositions or materials that are comprised of solid particles and are not dissolved in water or other solvents.

The term “volatile,” as used herein, unless otherwise specified, refers to those materials that are liquid under ambient conditions and which have a measurable vapor pressure at 25° C. These materials typically have a vapor pressure greater than about 0.01 mmHg, more typically from about 0.02 mmHg to about 20 mmHg, and an average boiling point typically less than about 250° C., more typically less than about 235° C.

The term “cosmetically acceptable”, as used herein, means that the product glides on smoothly during application, is non-irritating, and results in little or no visible residue (e.g., low residue performance) after application to the skin.

I. Structurants

A. Primary Structurants

The antiperspirant compositions contain a primary structurant. The primary structurant may be present in an amount of from about 1% to about 30%, more preferably from about 2% to about 20%, even more preferably from about 4% to about 16%, by weight of the composition.

The primary structurant is a petroleum wax, such as ozokerite. Commercially available ozokerite wax is a blend of petroleum-derived paraffin and microcrystalline waxes. Other types of commercially available petroleum waxes include but are not limited to Ceresin wax, paraffin wax, ader wax, earth wax, and petroleum derived microcrystalline wax.

Ozokerite waxes typically melt at the range of from about 60 to about 95° C. Ozokerite is also commonly known as ceresin, mineral wax, ozocerite, and white ceresin wax. Ozokerite is commercially available from suppliers including, but not limited to, Strahl & Pitsch, Inc; Frank B. Ross Company, Inc.

B. Secondary Structurants

The antiperspirant compositions of the present invention contain one or more secondary structurants. The secondary structurants may be present in an amount of from about 0.01% to about 25%, more preferably from about 0.5% to about 20%, even more preferably from about 1.0% to about 15%, by weight of the composition.

Suitable secondary structurants for use in the composition include structurants that are solids under ambient conditions, and preferably those of a crystalline material.

Secondary structurants for use in the antiperspirant composition include fatty alcohols, esters of fatty alcohols, fatty acids, amides of fatty acids, esters or ethers of fatty acids including triglycerides, non-petroleum based waxes, polyethylenes with molecular weight of from about 200 to about 1000 daltons; ethoxylated fatty alcohols, ethoxylated fatty acids, corresponding salts thereof, combinations thereof, and other crystalline suspending agents known or otherwise effective in providing the desired crystalline matrix within the antiperspirant composition. All such suspending agents preferably have a fatty alkyl moiety having from about 14 to about 60 carbon atoms, more preferably from about 18 to about 40 carbon atoms, and which may be saturated or unsaturated, substituted or unsubstituted, branched or linear or cyclic. Preferred fatty alkyl moieties are saturated, more preferably saturated and unsubstituted.

The term “substituted,” as used herein, refers to chemical moieties known or otherwise effective for attachment to suspending agents or other compounds. Such substitutes include those listed and described in C. Hansch and A. Leo, Substituent Constants for Correlation Analysis in Chemistry and Biology (1979). Examples of such substitutes include, but are not limited to, alkyl, alkenyl, alkoxy, hydroxy, oxo, nitro, amino, aminoalkyl (e.g., aminomethyl, etc.), cyano, halo, carboxy, alkoxyaceyl (e.g., carboethoxy, etc.), thiol, aryl, cycloalkyl, heteroaryl, heterocycloalkyl (e.g., piperidinyl, morpholinyl, pyrrolidinyl, etc.), imino, thioxo, hydroxyalkyl, aryloxy, arylalkyl, and combinations thereof.

Nonlimiting examples of suitable esters of fatty alcohols include tri-isostearyl citrate, ethyleneglycol di-12-hydroxystearate, tristearylcitrate, stearyl octanoate, stearyl heptanoate, trilaurylcitrate.

Suitable fatty alcohols may be used in the composition at concentrations preferably ranging from about 0.1% to about 8%, more preferably from about 2% to about 8%, even more preferably from about 3% to about 6%, by weight of the composition. The fatty alcohol suspending agents are also preferably saturated, unsubstituted, monohydric alcohols or combinations thereof, which have from about 14 to about 60 carbon atoms and a melting point preferably less than about 110° C. Specific examples of fatty alcohol suspending agents for use in the antiperspirant compositions that are commercially available include, but are not limited to, Unilin 550, Unilin 700, Unilin 425, Unilin 400, Unilin 350, and Unilin 325, all supplied by Petrolite.

Suitable ethoxylated suspending agents include, but are not limited to, Unithox 325, Unithox 400, and Unithox 450, Unithox 480, Unithox 520, Unithox 550, Unithox 720, Unithox 750, all of which are available from Petrolite.

Suitable fatty acid esters for use as crystalline structurants include, but are not limited to, ester waxes, monoglycerides, diglycerides, triglycerides and combinations thereof. Preferred are the glyceride esters. Nonlimiting examples of suitable ester waxes include, but are not limited to, stearyl stearate, stearyl behenate, palmityl stearate, stearyl octyldodecanol, cetyl esters, cetearyl behenate, behenyl behenate, ethylene glycol monostearate; ethylene glycol distearate and mixtures thereof, ethylene glycol dipalmitate, and beeswax. Examples of commercial ester waxes include Syncrowax ERL-C available from Croda; Kester waxes available from Koster Keunen; Crodamol SS available from Croda; and Demalcare SPS available from Rhone Poulenc.

Preferred are glyceryl tribehenate and other triglycerides, wherein at least about 75%, preferably about 100%, of the esterified fatty acid moieties of said other triglycerides each have from about 18 to about 36 carbon atoms, and wherein the molar ratio of glyceryl tribehenate to said other triglycerides is from about 20:1 to about 1:1, preferably from about 10:1 to about 3:1, more preferably from about 6:1 to about 4:1. The esterified fatty acid moieties may be saturated or unsaturated, substituted or unsubstituted, linear or branched, but are preferably linear, saturated, unsubstituted ester moieties derived from fatty acid materials having from about 18 to about 36 carbon atoms. The triglyceride gellant preferably has a melting point of less than about 110° C. Preferred concentrations of the triglyceride suspending agents in the antiperspirant composition range from about 3% to about 20%, more preferably from about 4% to about 10%, by weight of the composition. Specific examples of preferred triglyceride suspending agents include, but are not limited to, tristearin, tribehenate, behenyl palmityl behenyl triglyceride, palmityl stearyl palmityl triglyceride, hydrogenated vegetable oil, hydrogenated rapeseed oil, castor wax, fish oils, tripalmiten, Syncrowax HRC and Syncrowax HGL-C (Syncrowax is available from Croda, Inc.). Other suitable glycerides include, but are not limited to, and glyceryl stearate and glyceryl distearate.

Specific examples of preferred non-petroleum based waxes include, but are not limited to, beeswax; carnauba; candelilla; spermeceti wax; baysberry; synthetic waxes, such as Fisher-Tropsch waxes and non-petroleum based microcrystalline wax.

Some of the crystalline suspending agents suitable for use in the antiperspirant composition herein are also described in U.S. Pat. No. 5,552,136, (Motley), U.S. Pat. No. 5,976,514 (Guskey et al.), and U.S. Pat. No. 5,891,424 (Bretzler et al.).

II. Antiperspirant Active

The antiperspirant compositions of the present invention comprise a particulate antiperspirant active suitable for application to human skin. The concentration of antiperspirant active in the composition should be sufficient to provide the desired perspiration and odor control from the antiperspirant stick formulation selected.

The anhydrous antiperspirant stick compositions of the present invention comprise an antiperspirant active at concentrations of from about 0.5% to about 60%, and more preferably from about 5% to about 35%, by weight of the composition. These weight percentages are calculated on an anhydrous metal salt basis exclusive of water and any complexing agents such as, for example, glycine and glycine salts. The antiperspirant active as formulated in the composition are in the form of dispersed particulate solids having a preferred average particle size or equivalent diameter of less than about 100 microns, more preferably less than about 20 microns, and even more preferably less than about 10 microns.

The antiperspirant active for use in the anhydrous antiperspirant compositions of the present invention may include any compound, composition or other material having antiperspirant activity. More specifically, the antiperspirant actives may include astringent metallic salts, especially inorganic and organic salts of aluminum, zirconium and zinc, as well as mixtures thereof. Even more specifically, the antiperspirant actives may include aluminum-containing and/or zirconium-containing salts or materials, such as, for example, aluminum halides, aluminum chlorohydrate, aluminum hydroxyhalides, zirconyl oxyhalides, zirconyl hydroxyhalides, and mixtures thereof.

A. Aluminum Salts

Aluminum salts useful in the present invention include those that conform to the formula:
Al₂(OH)ₐCl_{b}.xH₂O
wherein a is from about 2 to about 5; the sum of a and b is about 6; x is from about 1 to about 6; and a, b, and x may have non-integer values. Particularly preferred are the aluminum chlorohydroxides referred to as “⅚ basic chlorohydroxide”, wherein a=5, and “⅔ basic chlorohydroxide”, wherein a=4.

Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980. Mixtures of aluminum salts are described in British Patent Specification 1,347,950, Shin et al., published Feb. 27, 1974.

B. Zirconium Salts

Preferred zirconium salts for use in the present invention include those which conform to the formula:
ZrO(OH)₂₋ₐClₐ.xH₂O
wherein a is from about 1.5 to about 1.87; x is from about 1 to about 7; and a and x may both have non-integer values.

These zirconium salts are described in Belgian Patent 825,146, Schmitz, issued Aug. 4, 1975. Preferred zirconium salts are those complexes that additionally contain aluminum and glycine, commonly known as “ZAG complexes”. These ZAG complexes contain aluminum chlorohydroxide and zirconyl hydroxy chloride conforming to the above-described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978.

III. Anhydrous Liquid Carriers

The antiperspirant compositions of the present invention may comprise an anhydrous liquid carrier at concentrations ranging from about 10% to about 90%, preferably from about 20% to about 80%, more preferably from about 30% to about 70%, by weight of the composition. Such concentrations will vary depending upon variables such as product form, desired product hardness, selection of other ingredients in the composition, and so forth. The anhydrous liquid carrier for use in the composition can be any anhydrous liquid that is known for use in personal care applications or is otherwise suitable for topical application to the skin. Preferred anhydrous liquid carriers include both volatile fluids and nonvolatile fluids.

A. Volatile Fluid

The antiperspirant composition of the present invention may further comprise a volatile fluid, preferably a volatile silicone carrier at concentrations ranging from about 20% to about 80%, and more specifically from about 30% to about 60%, by weight of the composition. The volatile silicone of the solvent may be cyclic, linear, and/or branched chain silicone. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976).

The volatile silicone is preferably a cyclic silicone having from about 3 to about 7 silicone atoms, and more preferably from about 5 to about 6, and still more preferably about 5 silicone atoms. Preferred are those which conform to the formula: 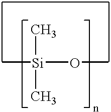
wherein n is from about 3 to about 7, preferably from about 5 to about 6, more preferably about 5. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof.

B. Non-Volatile Fluid

The antiperspirant composition of the present invention may further comprise a non-volatile fluid. These non-volatile fluids may be either non-volatile organic fluids or non-volatile silicone fluids.

1. Non-Volatile Organic Fluids

The antiperspirant composition of the present invention may further comprise non-volatile organic fluids. The non-volatile organic fluid can be present at concentrations ranging from about 1% to about 20%, and more preferably from about 2% to about 15%, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv.TM.), dipropylene glycol dibenzoate, PPG-15 stearyl ether benzoate and blends thereof (e.g. Finsolv TPP), neopentyl glycol diheptanoate (e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate. Many such other carrier liquids are disclosed in U.S. Pat. No. 6,013,248 (Luebbe et al.) and U.S. Pat. No. 5,968,489 (Swaile et al).

B. Nonvolatile Silicone Fluids

The antiperspirant compositions of the present invention may further comprise a non-volatile silicone fluid. The non-volatile silicone fluid is preferably a liquid at or below human skin temperature, or otherwise in liquid form within the anhydrous antiperspirant composition during or shortly after topical application. The concentration of the non-volatile silicone is from about 1% to about 15%, more preferably from about 2% to about 10%, by weight of the composition. Preferred are those nonvolatile silicone fluids which conform to the formula: 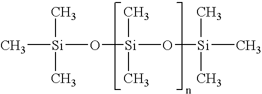
wherein n is greater than or equal to 1. These linear silicone materials will generally have viscosity values of from about 10 centistokes to about 100,000 centistoke, preferably less than about 500 centistoke, more preferably from about 5 centistoke to about 200 centistoke, even more preferably from about 10 centistoke to about 50 centistoke, as measured under ambient conditions.

Specific non limiting examples of suitable nonvolatile silicone fluids include Dow Corning 200, hexamethyldisiloxane, Dow Corning 225, Dow Corning 1732, Dow Corning 5732, Dow Corning 5750 (available from Dow Corning Corp.); and SF-96, SF-1066 and SF18(350) Silicone Fluids (available from G.E. Silicones).

Preferably, the low surface tension non-volatile solvent is selected from the group consisting of dimethicones, dimethicone copolyols, phenyl trimethicones, alkyl dimethicones, alkyl methicones, and mixtures thereof.

IV. Optional Ingredients

The antiperspirant compositions of the present invention may further comprise any optional material that is known for use in antiperspirant and deodorant compositions or other personal care products, or which is otherwise suitable for topical application to human skin. Nonlimiting examples of optional materials include dyes or colorants, emulsifiers, perfumes, distributing agents, antimicrobials, deodorant perfumes, pharmaceutical or other topical actives, preservatives, surfactants, processing aides such as viscosity modifiers, wash-off aids, and so forth. Examples of such optional materials are described in U.S. Pat. No. 4,049,792 (Elsnau); U.S. Pat. No. 5,019,375 (Tanner et al.); and U.S. Pat. No. 5,429,816 (Hofrichter et al.).

### Product Form

The antiperspirant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like, provided that the selected form contains all the essential elements as defined herein. Preferably, the antiperspirant compositions of the present invention are semi-solids or solids.

The antiperspirant products are generally stored in and dispensed from a suitable package or applicator device, such as a cream dispenser with perforated application domes, etc. These packages should be sufficiently closed to prevent excessive loss of volatiles prior to application.

### Method of Manufacture

The antiperspirant compositions of the present invention may be prepared by any known or otherwise effective technique, suitable for providing an anhydrous composition of the desired form and having the essential materials described herein. Many such techniques are described in the antiperspirant/deodorant formulation arts for the described product forms.

For example, the antiperspirant stick compositions can be formulated by mixing the volatile silicone and nonvolatile fluid materials under ambient conditions or under conditions sufficient to render the admixture fluid or liquid, and then adding the primary structurant and all secondary structurants to the mixture and heating the resulting mixture sufficiently to liquefy the added structurants, e.g., at approximately 85° C. for many wax solids, to form a single phase liquid. Antiperspirant actives can then be added to and dispersed throughout the heated, single-phase liquid before allowing the resulting combination to cool to approximately 78° C., at which point perfumes and similar other materials (if any) can be mixed into the combination. The combination can then be cooled to just above the solidification point of the suspending agent (e.g., typically about 60° C.), deposited into dispensing packages, and allowed to solidify under ambient conditions.

### Method of Use

The antiperspirant compositions of the present invention may be applied topically to the underarm or other suitable area of the skin in an amount effective to reduce or inhibit perspiration. Preferably, compositions of the present invention are applied in an amount ranging from at least about 0.1 gram to no more than about 20 grams, preferably no more than about 10 grams, more preferably no more than about 2 grams per axilla.

The composition is preferably applied to the underarm at least about one or two times daily, preferably once daily, to achieve effective antiperspirant reduction or inhibition over an extended period. The antiperspirant composition can also be applied every other day, or every third or fourth day, and then optionally to supplement application on off-days with other personal care products such as deodorants and/or conventional antiperspirant formulations.

Compositions of the present invention are preferably applied to skin, wherein the anhydrous liquid carrier leaves behind a skin-adhering and active-containing film. This film is positioned over the sweat ducts and resists flaking and/or rub-off, thereby being present through multiple perspiration episodes.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-5

| | I | II | III | IV | V |
|---|---|---|---|---|---|
| Al-ZR trichlorohydrex | 25.25 | 25.25 | 25.25 | 25.25 | 25.25 |
| glycinate (solid) | | | | | |
| Cyclopentasiloxane | 52.75 | 54.25 | 43.25 | 31.8 | 55.5 |
| Dimethicone (10 Cst) | 5.00 | 5.00 | 5.00 | | 5.00 |
| Petrolatum | 2.00 | 2.00 | 2.00 | 3.50 | 2.00 |
| Ozokerite | 11.00 | 11.70 | 14.30 | 7.25 | 5.00 |
| Stearyl Alcohol | 2.00 | | | 14.00 | |
| Polyethylene | 2.00 | | | | |
| Synrowax ERL-C | | 1.8 | 2.20 | | 1.25 |
| FINSOLV TPP | | | 3 | | |
| PPG-14 Butyl Ether | | | 5 | | |
| Castor Wax | | | | 3.50 | |
| Behenyl Alcohol | | | | 0.20 | |
| Mineral Oil | | | | 0.50 | |
| Talc | | | | 4.00 | 3.00 |
| Lexfeel 7 | | | | | 3.00 |

### Examples 6-10

| | VI | VII | VIII | IX | X |
|---|---|---|---|---|---|
| Al-ZR trichlorohydrex | 25.25 | 25.25 | 25.25 | 25.25 | 25.25 |
| glycinate (solid) | | | | | |
| Cyclopentasiloxane | 52.75 | 52.75 | 53.25 | 47.75 | 56.5 |
| Dimethicone (10 Cst) | | 5.00 | 5.00 | | 5.00 |
| Petrolatum | 2.00 | 2.00 | 2.00 | 2.00 | 3.00 |
| Ozokerite | 13.00 | 13.00 | 12.00 | 12.50 | 5.00 |
| Stearyl Alcohol | 2.00 | | 1.00 | 0.50 | |
| Polyethylene | | | 1.00 | | |
| Synrowax ERL-C | | | 0.50 | | |
| Glycerol Tribehenate | | | | 2.00 | |
| PPG-14 Butyl Ether | 5.00 | | | 1.00 | |
| Mineral Oil | | | | 1.00 | |
| Talc | | | | 3.00 | 4.00 |
| Lexfeel 7 | | | | 5.00 | |
| C18-36 Acid Glycol Ester | | 2.00 | | | 1.25 |

All documents cited in the Background, Summary of the Invention, and Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An anhydrous antiperspirant composition comprising:
a) a primary structurant;
b) a secondary structurant; and
c) an antiperspirant active;
wherein said primary structurant and said secondary structurant are any material which provides suspending, gelling, viscosifying, solidifying, thickening, and/or structuring properties to said anhydrous antiperspirant composition.

2. The composition of claim 1 wherein said primary structurant is a petroleum wax.

3. The composition of claim 2 wherein said petroleum wax is ozokerite wax.

4. The composition of claim 1 wherein said primary structurant is present in an amount of from about 1% to about 30% by weight of the composition.

5. The composition of claim 1 wherein said primary structurant is present in an amount of from about 2% to about 20% by weight of the composition.

6. The composition of claim 1 wherein said primary structurant is present in an amount of from about 4% to about 16% by weight of the composition.

7. The composition of claim 1 further comprising an anhydrous liquid carrier.

8. The composition of claim 1 further comprising an additional ingredient selected from the group consisting of dyes or colorants, emulsifiers, perfumes, distributing agents, antimicrobials, deodorant perfumes, pharmaceutical or other topical actives, preservatives, surfactants, processing aides such as viscosity modifiers, and wash-off aids.

9. The composition of claim 1 wherein said composition is in the product form selected from the group consisting of liquid, solid, and semi-solid.

10. The composition of claim 9 wherein said product form is a solid.

11. The composition of claim 9 wherein said product form is a semi-solid.

12. A method of using the composition of claim 1 comprising the step of applying from about 0.1 gram to about 2.0 grams of said composition to each axilla.
